(19) United States
(12) Patent Application Publication
(10) Pub. No.: US 2019/0320978 A1
(43) Pub. Date: Oct. 24, 2019

(54) HELMET AND METHOD OF CONTROLLING THE SAME

(71) Applicants: Hyundai Motor Company, Seoul (KR); Kia Motors Corporation, Seoul (KR)

(72) Inventors: Jeong-Eom Lee, Yongin (KR); Dong-Seon Chang, Hwaseong (KR); Dongsoo Shin, Suwon (KR); Seona Kim, Seoul (KR)

(21) Appl. No.: 16/164,993

(22) Filed: Oct. 19, 2018

(30) Foreign Application Priority Data
Apr. 20, 2018 (KR) 10-2018-0045996

(51) Int. Cl. A61B 5/00; A61B 5/0482; A42B 3/04; A61B 5/117; A42B 3/22; A61B 5/04

(52) U.S. Cl. CPC A61B 5/6803; A61B 5/0482; A42B 3/0466; A61B 5/741; A61B 5/7246; A61B 5/117; A61B 2560/0475; A42B 3/222; A61B 5/7445; A61B 5/4803; A61B 5/0006; A61B 5/04012; A61B 2560/0209; A61B 5/7455

(57) ABSTRACT

A helmet may include: a brain wave detector configured to detect a brain wave of a user; a sound input configured to receive sound; a sound output configured to output sound; and a controller configured to, in response to detecting the brain wave of the user, compare the detected brain wave with a previously stored brain wave, to activate a dialogue mode when the detected brain wave matches the previously stored brain wave, to recognize a speech from the sound received by the sound input, to generate a dialogue speech based on the recognized speech, and to control the sound output so as to output the generated dialogue speech.

### CROSS-REFERENCE TO RELATED APPLICATION

This application is based on and claims the benefit of priority under 35 U.S.C. § 119 to Korean Patent Application No. 10-2018-0045996, filed on Apr. 20, 2018 in the Korean Intellectual Property Office, the disclosure of which is incorporated herein by reference as if fully set forth herein.

### BACKGROUND

### 1. Technical Field

The present disclosure relates to a helmet and a method for controlling the same and, more particularly, to a helmet for protecting the head of a user and outputting information corresponding to a biometric signal of the user, and a method of controlling the same.

### 2. Description of the Related Art

Due to the increase in environmental regulations and large cities, small one-person vehicles are increasingly popular, a concept referred to as “personal mobility.” Personal mobility devices may include small-sized vehicles for middle/short-range travel that combine electric charging technology and power technology. Use of such devices is also referred to as “smart mobility” or “micro mobility.”

The personal mobility devices operate on electricity, thus emitting no pollutants. In addition, personal mobility devices are easy to carry and can solve problems relating to traffic congestion and lack of parking.

While on the move using the personal mobility device, accidents may occur due to a collision with another object or a slip of a wheel. In order to minimize the damage of the user's head caused by an unexpected accident, the use of helmets is increasing.

Recently, the increased use of helmets has caused numerous technological developments in which a user is provided with various functions through a helmet by incorporating peripheral devices such as a camera, a brain wave sensor, a speaker, and the like. These functions include, for example, acquiring a user's biometric signal through image capturing and brain wave detection, outputting information to a user for encouraging safe driving on the basis of the obtained biometric signal, playing music through a speaker, and the like.

### SUMMARY

It is an aspect of the present disclosure to provide a helmet capable of performing a dialogue with a user and a method of controlling the same.

It is also an aspect of the present disclosure to provide a helmet capable of recognizing a brain wave, transmitting an ignition-on command to a personal mobility device, and waking up a dialogue controller, and a method of controlling the same.

It is further an aspect of the present disclosure to provide a helmet capable of recognizing a user's emotion on the basis of at least one of a brain wave, a facial expression, and a speech of a user, changing a control right of a personal mobility device on the basis of the recognized emotion, and controlling output of a dialogue controller, and a method of controlling the same.

Additional aspects of the disclosure will be set forth in part in the description which follows and, in part, will be apparent from the description, or may be learned by practice of the disclosure.

In accordance with embodiments of the present disclosure, a helmet may include: a brain wave detector configured to detect a brain wave of a user; a sound input configured to receive sound; a sound output configured to output sound; and a controller configured to, in response to detecting the brain wave of the user, compare the detected brain wave with a previously stored brain wave, to activate a dialogue mode when the detected brain wave matches the previously stored brain wave, to recognize a speech from the sound received by the sound input, to generate a dialogue speech based on the recognized speech, and to control the sound output so as to output the generated dialogue speech.

The helmet may further include a dialogue controller that enables a dialogue with the user. The controller may transmit a wake-up command to the dialogue controller when activating the dialogue mode.

The previously stored brain wave may include a brain wave generated from a brain of the user before an utterance of a wake word for calling the dialogue controller, the wake word being a target for the dialogue.

The previously stored brain wave may include a brain wave generated from a brain of the user during an object association process for performing the dialogue before an utterance of the user is made.

The helmet may further include a communicator configured to perform communication with a personal mobility device, wherein the controller may transmit an ignition-on command to the personal mobility device when the detected brain wave matches the previously stored brain wave.

The controller may be configured to, in response to receiving a registration command for an authentication brain wave of the personal mobility device, control the sound output so as to output a speech corresponding to an object association request, and to register a brain wave recognized from a point of time when the speech corresponding to the object association request is output as the authentication brain wave.

The helmet may further include an image input configured to receive an image of the user, and the controller may obtain a facial image of the user from the received image, recognize a facial expression of the user from the obtained facial image, recognize an emotional state of the user based on the recognized facial expression, and limit a target speed of the personal mobility device based on the recognized emotional state.

The controller may recognize the emotional state of the user using the brain wave of the user and a speech of the user.

The helmet may further include a communicator configured to perform communication with a terminal, and the controller, when the recognized speech corresponds to a function control command of the terminal, may be further configured to transmit the function control command to the terminal, and in response to receiving function execution information of the terminal, may be further configured to control output of the received function execution information.

The helmet may further include a vibrator configured to generate a vibration in response to the transmission of the function control command and the reception of the function execution information.

The helmet may further include: a main body in which the brain wave detector, the sound input, and the sound output are provided; and a visor configured to be rotated about a gear axis of the main body.

The helmet may further include an image output provided in the visor, the image output configured to output an image.

The helmet may further include an image output configured to project an image to the visor such that the image is reflected on the visor.

The controller, in response to receiving a registration command for a wake word brain wave for activating the dialogue mode, may be further configured to output a speech corresponding to an object association request which corresponds to a wake word, and to register a brain wave recognized from a point of time when the speech corresponding to the object association request is output as the wake word brain wave.

Furthermore, in accordance with embodiments of the present disclosure, a helmet may include: a brain wave detector configured to detect a brain wave of a user; a sound input configured to receive sound; a sound output configured to output sound; a communicator configured to communicate with an external device that performs a dialogue; and a controller configured to, in response to detecting the brain wave of the user, compare the detected brain wave with a previously stored brain wave, to activate a dialogue mode when the detected brain wave matches the previously stored brain wave, to transmit a signal corresponding to the sound received by the sound input to the external device, and to control the sound output so as to output a signal of a speech transmitted from the external device.

The previously stored brain wave may include a brain wave generated from a brain of the user before an utterance of a wake word for calling the dialogue controller, the wake word being a target for the dialogue.

The controller may be further configured to, in response to receiving a wake word for activating the dialogue mode, output a speech corresponding to an object association request which corresponds to the wake word, and to register a brain wave recognized from a point of time when the speech corresponding to the object association request is output as a wake word brain wave.

The previously stored brain wave may include a brain wave generated from a brain of the user during an object association process for performing the dialogue before an utterance of the user is made.

Furthermore, in accordance with embodiments of the present disclosure, a method of controlling a helmet may include: detecting a brain wave of a user; comparing the detected brain wave with a previously stored brain wave; activating a dialogue mode when the received brain wave matches the previously stored brain wave; recognizing a speech from sound received by a sound input of the helmet; generating a dialogue speech based on the recognized speech; and outputting the generated dialogue speech through a sound output of the helmet.

The activation of a dialogue mode may include: transmitting a wake-up command to a dialogue controller that is configured to enable a dialogue with the user.

The method may further include transmitting an ignition-on command to a personal mobility device when the detected brain wave matches the previously stored brain wave.

The method may further include: in response to receiving a registration command for an authentication brain wave of the personal mobility device, outputting a speech corresponding to an object association request; and registering a brain wave recognized from a point of time when the speech corresponding to the object association request is output as the authentication brain wave.

The method may further include: in response to receiving a registration command for a wake word brain wave for activating the dialogue mode, outputting a speech corresponding to an object association request which corresponds to a wake word; and registering a brain wave recognized from a point of time when the speech corresponding to the object association request is output as the wake word brain wave.

The method may further include: when the recognized speech is a function control command of a terminal, transmitting the function control command to the terminal, and in response to receiving function execution information of the terminal, outputting the received function execution information.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a view illustrating an external appearance of a helmet according to embodiments of the present disclosure;

FIG. 2 is a control block diagram of a helmet according to embodiments of the present disclosure;

FIG. 3 is a detailed block diagram of a controller of a helmet according to embodiments of the present disclosure;

FIG. 4 is a flowchart showing registration of brain waves for authentication of a helmet and a personal mobility device according to embodiments of the present disclosure;

FIG.5 is a control flowchart for controlling the ignition of a personal mobility device using a helmet according to embodiments of the present disclosure;

FIG. 6 is a control flowchart for controlling operations of a personal mobility device using a helmet according to embodiments of the present disclosure;

FIG. 7 is a flowchart showing a procedure in which a call-word brain wave is registered in a dialogue mode according to embodiments of the present disclosure;

FIG. 8 is a flowchart of entry of a dialogue mode when a dialogue mode is performed using a helmet according to embodiments of the present disclosure;

FIG. 9 is another control block diagram of a helmet according to embodiments of the present disclosure; and

FIG. 10 is another control flowchart of a helmet according to embodiments of the present disclosure.

It should be understood that the above-referenced drawings are not necessarily to scale, presenting a somewhat simplified representation of various preferred features illustrative of the basic principles of the disclosure. The specific design features of the present disclosure, including, for example, specific dimensions, orientations, locations, and shapes, will be determined in part by the particular intended application and use environment.

### DETAILED DESCRIPTION OF THE EMBODIMENTS

Hereinafter, embodiments of the present disclosure will be described in detail with reference to the accompanying drawings. As those skilled in the art would realize, the described embodiments may be modified in various different ways, all without departing from the spirit or scope of the present disclosure.

Like numerals refer to like elements throughout the specification. Not all elements of embodiments of the present disclosure will be described, and description of what are commonly known in the art or what overlap each other in the embodiments will be omitted. The terms as used throughout the specification, such as “˜part”, “˜module”, “˜member”, “˜block”, etc., may be implemented in software and/or hardware, and a plurality of “˜parts”, “˜modules”, “˜members”, or “˜blocks” may be implemented in a single element, or a single “˜part”, “˜module”, “˜member”, or “˜block” may include a plurality of elements.

It will be further understood that the term “connect” or its derivatives refer both to direct and indirect connection, and the indirect connection includes a connection over a wireless communication network.

Further, when it is stated that one member is “on” another member, the member may be directly on the other member or a third member may be disposed therebetween.

The term “include (or including)” or “comprise (or comprising)” is inclusive or open-ended and does not exclude additional, unrecited elements or method steps, unless otherwise mentioned.

It will be understood that, although the terms first, second, etc. may be used herein to describe various elements, these elements should not be limited by these terms. These terms are only used to distinguish one element from another.

It is to be understood that the singular forms “a,” “an,” and “the” include plural references unless the context clearly dictates otherwise.

Reference numerals used for method steps are just used for convenience of explanation, but not to limit an order of the steps. Thus, unless the context clearly dictates otherwise, the written order may be practiced otherwise.

Additionally, it is understood that one or more of the below methods, or aspects thereof, may be executed by at least one controller. The term “controller” may refer to a hardware device that includes a memory and a processor. The memory is configured to store program instructions, and the processor is specifically programmed to execute the program instructions to perform one or more processes which are described further below. The controller may control operation of units, modules, parts, or the like, as described herein. Moreover, it is understood that the below methods may be executed by an apparatus comprising the controller in conjunction with one or more other components, as would be appreciated by a person of ordinary skill in the art.

Hereinafter, embodiments of the present disclosure will be described with reference to the accompanying drawings.

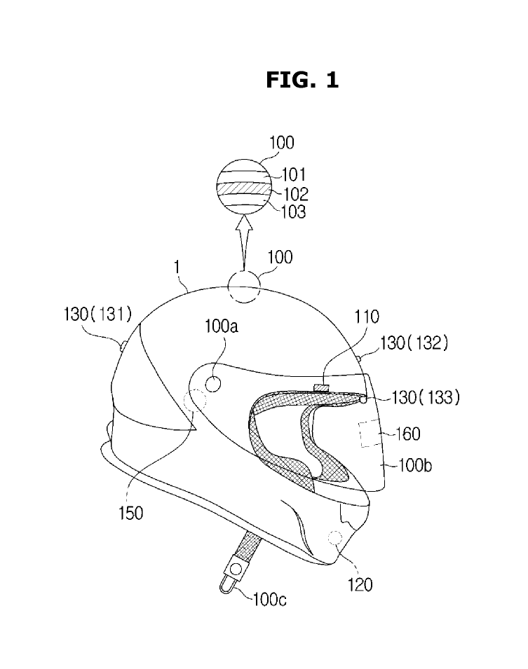

FIG. 1 is an external view of a helmet according to embodiments of the present disclosure. It is understood that the external view of the helmet as described herein and illustrated in FIG. 1 is provided for demonstration purposes only and thus does not limit the scope of the present disclosure.

A helmet 1 is a device worn on a user's head to protect the user's head. The types of helmets may be divided into personal mobility device helmets, motorcycle helmets, bicycle helmets, board helmets, skiing helmets, skating helmets, and safety helmets depending on intended uses. The following description is made in relation to a helmet for a personal mobility device according to embodiments of the present disclosure. Examples of the personal mobility device include, but are not limited to, electric scooters, mopeds, skateboards, skates, hoverboards, bicycles, unicycles, wheelchairs, and the like, as well as other electrically powered walking aids.

The helmet 1 includes a main body 100 capable of receiving and covering the head of a user. The main body 100 includes an outer shell 101, a shock absorber 102, and an inner pad 103.

The outer shell 101 primarily absorbs a shock upon collision with an object.

The shock absorber 102 is provided between the outer shell 101 and the pad 103, and secondarily absorbs shock to reduce the amount of impact transmitted to the user. The shock absorber 102 may include a styrofoam (EPS) layer which has lightweight, excellent shock absorption, ease of mold, and exhibits a stable performance regardless of whether the temperature is high or not.

The pad 103 distributes the weight of the helmet and improves the wearing sensation. That is, the pad is formed of soft and elastic material.

The helmet 1 further includes a visor 100b (also, referred to as a shield) mounted on the main body 100 and movably mounted on the main body 100 about an axis of a gear 100a and a fastening member 100c for fixing the main body 100 to the user's head to prevent the main body 100 from being separated from the user's head.

The visor 100b protects the user's face at a time of the collision and secures the view of the user while on the move. The visor 100b is formed of transparent material, and may include a film for glare control and UV blocking. In addition, the visor 100b may be implemented as a visor, a color or transparency of which is automatically changed according to illumination using a chemical or electrical method.

The fastening member 100c may be fastened or detached by a user. The fastening member 100c is provided to come into contact with the jaw of the user and thus is formed of material having excellent hygroscopicity and durability.

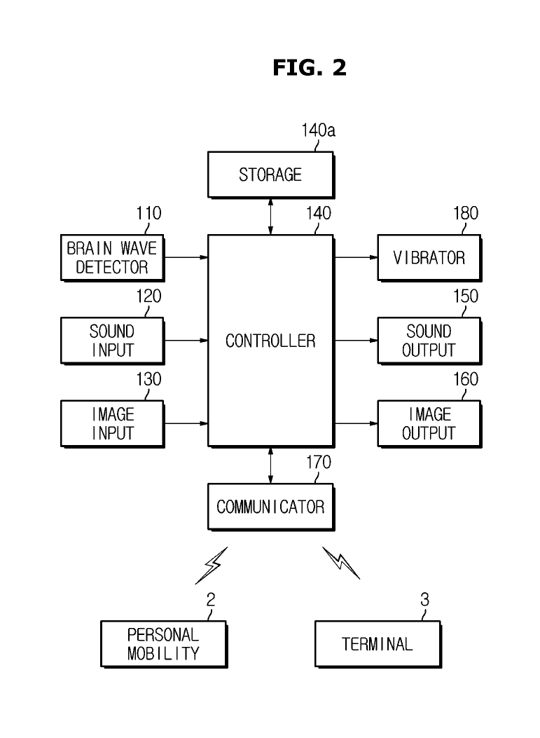

The helmet 1 includes a brain wave detector 110, a sound input 120, an image input 130, a controller 140 (see FIG. 2), a storage 140a, a sound output 150, an image output 160, a communicator 170, and a vibrator 180.

The brain wave detector 110 detects a user's brain wave. The brain wave detector 110 is positioned inside the main body 100 of the helmet so as to make contact with the head of the user. The brain wave detector 110 makes contact with the head (i.e., the forehead) adjacent to the frontal lobe responsible for memory and thinking skill, and detects brain waves generated in the frontal lobe.

The sound input 120 receives the user's speech. The sound input 120 includes a microphone. The sound input 120 may be provided in the main body 100 of the helmet, at a position adjacent to the user's mouth.

The image input 130 receives an image of a surrounding environment of the user. The image input 130 may include a first image input 131 for receiving a rear view image of the user, and may further include second image input 132 for receiving a front view image of the user, and may further include a third image input 133 for receiving a facial image of the user.

The first image input 131 may be provided on the rear side of the main body of the helmet, the second image input 132 may be provided on the front surface of the main body of the helmet, and the third image input 133 may be provided on the front side of the main body of the helmet with an image capturing surface directed to the face of the user.

In addition, the helmet 1 may further include a manipulator (not shown) for receiving power on/off commands, a registration command of an authentication brain wave, and a registration command of a wake word brain wave, and may further include a power source unit (mot shown) for supplying the respective components with driving power. Here, the power source unit may be a rechargeable battery.

The controller 140 (see FIG. 2) controls operations of the sound output 150, the image output 160, and the vibrator 170 on the basis of at least one of the brain waves detected by the brain wave detector 110, the sound inputted to the sound input 120, and the image inputted to the image input 130, and performs a dialogue with the user. The configuration of the controller 140 will be described later.

The sound output 150 outputs a dialogue speech as a sound in response to a control command of the controller 140. Here, the dialogue speech includes a response speech corresponding to a user's utterance and a query speech voice. The sound output 150 outputs a sound corresponding to sound information outputted from a terminal 3. The sound output 150 may include speakers that may be disposed on the left and right sides of the main body of the helmet, respectively. That is, the sound output 150 may include speakers provided at position adjacent to the user's ears.

The image output 160 outputs an image corresponding to a control command of the controller 140. The image output 160 may display a rear side image of the user inputted to the first image input 131. The image output 160 may display music information outputted on the terminal 3, navigation information outputted on the terminal 3, a message received by the terminal 3, and speed information of a personal mobility device 2.

The image output 160 may display a registration image for registration of a user of the helmet, an authentication image for authentication of the helmet and the personal mobility device, and a wake word image corresponding to an utterance of a wake word. Here, the registration image, the wake word image, and the authentication image may be an object that the user can come up with, an image of the object, or a text of the object.

The image output 160 may include a display for displaying an image. Here, the display may be a display panel provided in the visor 100b.

The image output 160 may be provided on the main body of the helmet and may project an image to the visor such that an output image is reflected on the visor 100b during output of an image. The image output 160 may be a Head-Up Display.

The communicator 170 performs communication with at least one of the personal mobility device 2 and the terminal 3. The communicator 170 transmits an ignition-on command to the personal mobility device 2 in response to a control command of the controller 140. In addition, the communicator 170 may transmit an ignition-off command to the personal mobility device 2, and may transmit a speed control command and a target speed to the personal mobility device 2.

The communicator 170 may transmit a function control command to the terminal 3 in response to a control command of the controller 140. Here, the function control command may include a call reception and transmission command, a message sending command, a music play command, a navigation control command, and the like. The communicator 170 may transmit function execution information transmitted from the terminal to the controller 140.

The communicator 170 may include at least one component that enables communication with an external device, for example, at least one of a short-range communication module, a wired communication module, and a wireless communication module. Here, the external device may include at least one of the personal mobility device 2 and the terminal 3.

The short-range communication module may include various short range communication modules that transmits and receives signals using a wireless communication network in a short range area, such as a Bluetooth module, an infrared communication module, an Radio Frequency Identification (RFID) communication module, a Wireless Local Access Network (WLAN) communication module, an NFC communication module, a Zigbee communication module, and the like.

The wired communication module may not only include various wired communication modules, such as a local area network (LAN) module, a wide area network (WAN) module or a value added network (VAN), but also various cable communication modules, such as Universal Serial Bus (USB) module, a high definition multimedia interface (HDMI), a digital visual interface (DVI), a recommended standard 232 (RS-232), a power line communication, or a plain old telephone service (POTS), and the like.

The wireless communication module includes wireless communication modules that support various communication methods, such as a WiFi module, a Wireless broadband (Wibro) module, a Global System for Mobile Communication (GSM) module, a Code Division Multiple Access (CDMA) module, a Wideband Code Division Multiple Access (WCDMA) module, a universal mobile telecommunications system (UMTS) module, Time Division Multiple Access (TDMA) module, Long Term Evolution (LTE) module, and the like.

The vibrator 180 generates vibration serving as feedback information corresponding to a function execution of the terminal 3 corresponding to a control command of the controller 140. For example, the vibrator 180 may generate vibration when message reception information is received, generate vibration when message transmission is completed, and generate vibration when a call incoming is made.

The helmet may further include a wear detector (not shown) for detecting whether or not a user wears the helmet. The wear detector may include a switch provided on the main body and configured to be turned on based on wearing. The wear detector may be a connection detector that generates a signal corresponding to whether a fastening member is coupled or not.

The personal mobility device may further include a departure detector (not shown) for detecting getting on and getting off. For example, the departure detector may include at least one of a weight detector, a pressure detector, and a switch.

The controller 140 is configured to, in response to receiving a registration command of an authentication brain wave, controls the sound output to output a speech of an object association request to a user such that an object for authentication with the personal mobility device is recalled, recognize a brain wave detected by the brain wave detector for a predetermined time period from a point of time when the speech corresponding to the object association request is output, and register the recognized brain wave as an authentication brain wave. The registering of the authentication brain wave includes storing the recognized brain wave as an authentication brain wave in the storage.

The controller 140 may be configured to, in response to receiving a registration command of an authentication brain wave for authentication of the personal mobility device, recognize a brain wave detected by the brain wave detector for a predetermined time period from a point of time when the registration command of an authentication brain wave is received, and register the recognized brain wave as an authentication brain wave. The recognizing of brain waves include recognizing a signal of a brain wave detected by the brain wave detector and recognizing a particular point and a particular pattern of the recognized signal.

The basic features of brain waves generated from wearing a helmet may be similar between users, but the users each have a distinct feature due to a variety of feelings and thoughts felt while wearing the helmet. Accordingly, the controller 140 recognizes a distinct feature that appears in at least one of an amplitude, a waveform, and a frequency of the brain waves generated when the helmet is worn. That is, the controller 140 identifies the amplitude, waveform, and frequency of the user's brain waves generated from wearing the helmet and recognizes at least one of a particular pattern and a particular value appearing in at least one of the amplitude, waveform, and frequency of the identified brain waves.

The registration command of an authentication command for authentication with the personal mobility device may include input via a user's speech, or input of an operation command through the manipulator. That is, when a registration command of a wearing brain wave for authentication with the personal mobility device is received, the controller 140 may recognize a brain wave detected by the brain wave detector 110 for a predetermined period of time from a point of time when the registration command of a wearing brain wave is received, and register the recognized brain wave as a wearing wave.

The controller 140 may receive brain waves detected by the brain wave detector 110 and compare the received brain wave with an authentication brain wave of the terminal, and may unlock the terminal when the received brain wave matches the authentication brain wave of the terminal. The controller 140 is configured to, in response to receiving a registration command of a user registration brain wave, control the sound output to output a speech of an object association request to a user such that an object for user registration is recalled, recognize a brain wave detected by the brain wave detector for a predetermined time period from a point of time when the speech corresponding to the object association request is output, and register the recognized brain wave as an authentication brain wave.

The controller 140 is configured to, in response to receiving a registration command of a helmet wearing brain wave, determine whether wear detection information is received from the wear detector, and in response to determination that wear detection information is received, recognize a brain wave detected by the brain wave detector for a predetermined time period from a point of time when the wear detection information is received, and register the recognized brain wave as a wear brain wave. The registering of a wear brain wave includes storing the wear brain wave in the storage.

The controller 140 is configured to, in response to receiving a registration command of a wake word for performing a dialogue mode, controls the sound output to output a speech of an object association request to a user such that an object for the wake word is recalled, recognize a brain wave detected by the brain wave detector for a predetermined time period from a point of time when the speech corresponding to the object association request is output, and register the recognized brain wave as a wake word brain wave. The object for a wake word may be an image of a certain object, a text of a certain object, and a name of a dialogue controller (or a dialogue system).

The controller 140 is configured to, in response to receiving a registration command of a wake word brain wave for performing a dialogue mode, controls the sound output to output a speech of a wake word utterance request, recognize a brain wave detected by the brain wave detector for a predetermined time period from a point of time when the speech of the wake word utterance request is output, and register the recognized brain wave as an authentication brain wave. The controller 140 recognizes brain waves generated from a point of time before an utterance for performing a dialogue mode is performed until the utterance is performed as wake word brain waves, and registers the recognized brain waves as the wake word brain waves. Here, the registering of wake word brain waves includes storing wake word brain waves in the storage.

The controller 140, for the registration of wake word brain waves, repeats the process of recognizing wake word brain waves a predetermined number of times, recognizes a common point between signals of the wake word brain waves recognized by the predetermined number of times, and recognizes the wake word brain wave. The controller 140 receives a brain wave detected by the brain wave detector 110, compares the received brain wave with the wake word brain wave, and controls entry into the dialogue mode if the received brain wave matches the wake word brain wave. Herein, the controlling of the entry into the dialogue mode includes waking up the dialogue controller.

The controller 140 recognizes a speech in sound inputted to the sound input during the execution of the dialogue mode, and generates at least one of a response speech and a query speech on the basis of the recognized speech and controls the sound output to output the generated speech. The controller 140 determines whether the recognized speech is a function control command of the terminal 3, and controls the communicator 140 to transmit the determined function control command to the terminal when it is determined that the speech is a function control command of the terminal.

The controller 140, for the communication between the personal mobility device and the terminal, may confirm identification information of the personal mobility device and may confirm identification information of the terminal. The controller 140 may determine a road condition based on an image inputted through the first and second image inputs and output the determined road condition using a speech.

The controller 140 receives a brain wave detected by the brain wave detector 110, compares the received brain wave with the authentication brain wave, and transmits an ignition-on command to the personal mobility device when the received brain wave matches the authentication brain wave. The controller 140 controls at least one of the sound output and the image output to output an ignition- on information and serviceability information of the personal mobility device when the received brain wave matches the authentication brain wave, and controls at least one of the sound output and the image output to output user authentication error notifying that the user is not an authenticated user and unserviceability information of the personal mobility device when the received brain wave does not match the authentication brain wave.

The controller 140 may compare the received brain wave with the user registration brain wave, and perform authentication with the personal mobility device when the received brain wave matches the user registration brain wave, and output unregistered user guide information notifying that the user is not a registered user when the received brain wave does not match the user registration brain wave. The controller 140, for the authentication with the personal mobility device, may compare identification information transmitted from the personal mobility device with identification information stored in the storage 140a and compare the received brain wave with the authentication brain wave when the two pieces of identification information match.

The controller 140 may activate the brain wave detector 110 when wear detection information is received from the wear detector, receive a brain wave detected by the brain wave detector, and compare the received brain wave with the authentication brain wave. The controller 140 may receive the brain wave detected by the brain wave detector 110 and compare the received brain wave with the wear brain wave, and when the received brain wave matches the wear brain wave, transmit an ignition-on command to the personal mobility device, and control at least one of the sound output and the image output to output ignition-on information of the personal mobility device.

In addition, the controller 140 controls at least one of the sound output and the image output to output safety information that notifies the user of the danger associated with non-wearing of the helmet when the received brain wave does not match the wear brain wave. The controller 140 may receive a brain wave detected by the brain wave detector 110, compare the received brain wave with the authentication brain wave, and when the received brain wave matches the authentication brain wave, compare the received brain wave with the wear brain wave, and when the received brain wave matches the wear brain wave, transmit an ignition-on command to the personal mobility device.

The controller 140 may transmit an ignition-on command to the personal mobility device 2 when the received brain wave matches the authentication brain wave and a riding detection signal is received from the personal mobility device. The controller 140 may transmit an ignition-off command to the personal mobility device 2 when the received brain wave matches the authentication brain wave and a departure signal is received from the personal mobility device.

The controller 140 may receive a brain wave detected by the brain wave detector 110, compare the received brain wave with the wear brain wave, and when the received brain wave does not match the wear brain wave, transmit an ignition-off command to the personal mobility device 2. The controller 140 may transmit an ignition-off command to the personal mobility device 2 and also may output at least one of the sound output and the image output to output ignition-off guide information when the received brain wave does not match the wear brain wave more for a reference time or longer. In this manner, the user is informed of the ignition-off of the personal mobility device before the transmission of ignition-off command to the personal mobility device 2.

The controller 140 controls the communicator 170 to transmit the ignition-on command and the ignition-off command to the personal mobility device. The controller 140 recognizes a facial expression of the user based on the image inputted through the third image input during the execution of the dialogue mode and recognizes the user's emotion based on the recognized facial expression.

The controller 140 may recognize the tone and the way of talking the recognized speech recognized during the execution of the dialogue mode, and recognize the user's emotion based on the tone and the way of talking of the recognized speech, or may recognize the user's emotion based on the recognized brain wave. That is, the controller 140 recognizes the user's emotion based on at least one of the image, the speech, and the brain waves, and changes the tone and voice of the speech outputted in the dialogue mode on the basis of the recognized emotion.

The controller 140 recognizes the user's emotion based on at least one of the image, the speech, and the brain waves, and determines an object to be assigned a control right of the personal mobility device 2 on the basis of the recognized emotion. That is, the controller 140 changes the control right of the personal mobility device from the user to the personal mobility device or to the helmet when the user's feeling is anger, irritation, tension, sadness, excitement or frustration.

When the personal mobility device is capable of autonomous driving, the controller 140 changes the control right of the personal mobility device from the user to the personal mobility device 2. In addition, when the personal mobility device is not capable of autonomous driving, the controller 140 changes the control right of the personal mobility device from the user to the helmet 1. At this time, the controller 140 restricts the maximum driving speed of the personal mobility device to a target speed, recognizes signal lamps, obstacles and the like based on image information, adjusts the driving speed of the personal mobility device based on the recognized information, such that the personal mobility device is driven at the target speed or below.

At least one component may be added or deleted corresponding to the performance of the components of the helmet 1 shown in FIG. 2. It will be readily understood by those skilled in the art that the relative positions of the components may be changed corresponding to the performance or structure of the system.

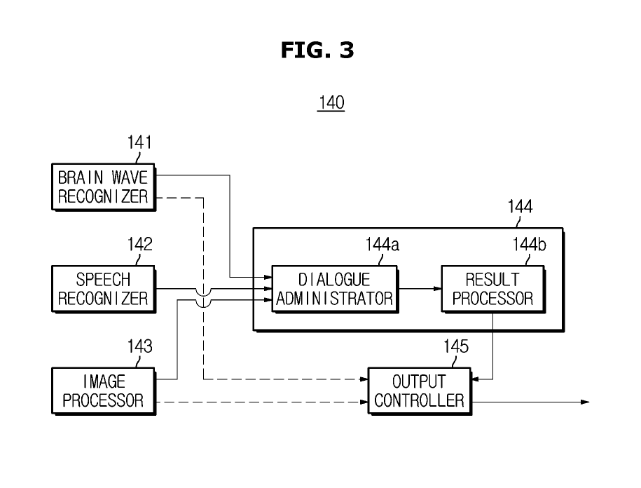

FIG. 3 is a detailed block diagram of a controller of a helmet according to embodiments of the present disclosure. As shown in FIG. 3, the controller 140 may include a brain wave recognizer 141, a speech recognizer 142, an image processor 143, a dialogue controller 144, and an output controller 145.

The brain wave recognizer 141 receives a brain wave detected by the brain wave detector 110 and recognizes the received brain wave signal, thereby recognizing whether the received brain wave is an authentication brain wave, a wear brain wave, or a wake word brain wave. The brain wave recognizer 141, in response to determining that the received brain wave is a wake word brain wave, transmits received information of the wake word brain wave to the dialogue controller 144. The brain wave recognizer 141, in response to determining that the received brain wave is a wake word brain wave, transmits a wakeup command to the dialogue controller 144 and transmit a wakeup command to the sound input 120 and the sound output 150. The brain wave recognizer 141, in response to determining that the received brain wave is an authentication brain wave signal or a wear brain wave signal, may transmit received information of the authentication brain wave signal or wear brain wave to the output controller 145.

The speech recognizer 142 recognizes the speech from the sound inputted to the sound input 120 and transmits information about the recognized speech to the dialogue controller 144.

The image processor 143 performs pre-processing and post-processing on the images inputted from the first, second, and third image inputs, and transmits the image-processed image information to the dialogue controller 144 and the output controller 145.

The dialogue controller 144 activates a dialogue mode for which the wake-up command is received, and controls output of a response speech corresponding to the recognized speech, the recognized brain wave, and the image processed image. The dialogue controller 144 may include a dialogue administrator 144a and a result processor 144b.

The dialogue administrator 144a recognizes the intention of the user from the recognized speech that is identified through natural language understanding, recognizes a surrounding circumstance of the user based on the recognized brain wave and the surrounding image, determines an action corresponding to the recognized intention of the user and the current surrounding circumstance, and transmits the determined action to the result processor 144b.

The result processor 144b generates a response speech and a query speech of the dialogue that are required to perform the received action on the basis of the received action, and transmits information about the generated speech to the output controller 145. Here, the response speech and the query speech of the dialogue may be output as a text, image or audio. The result processor 144b may output a control command of an external device, and when the control command is output, the result processor 144b may transmit the control command to the external device corresponding to the output command. The output controller 145 controls output of speech information of the dialogue controller, control information of the personal mobility device, control information of the terminal, and image information of the first and second image inputs. More specifically, the output controller 145, in response to receiving information about a response speech and a query speech from the result processor 144b of the dialogue controller, may generate a speech signal based on the received information, and control the sound output to output the generated speech signal.

The output controller 145 may control the image output 160 to output the image processed by the image processor 143. The output controller 145, in response to receiving control information of the external device, controls the communicator 170 to transmit the control command to the external device on the basis of the received control command, and controls the communicator 170 to receive information about the external device from the external device. For example, the output controller 145, in response to receiving a driving control command of the personal mobility device, may transmit the driving control command to the personal mobility device received, and in response to receiving a function control command of the terminal 3, may transmit the function control command to the terminal.

The output controller 145, in response to function execution information from the terminal, may control the sound output and the image output to output the received function execution information in the form of image and sound. The output controller 145 may transmit an ignition-on/off command to the personal mobility device on the basis of the received brain waves.

Each of the components shown in FIG. 3 refers to a software component and/or a hardware component such as a Field Programmable Gate Array (FPGA) and an Application Specific Integrated Circuit (ASIC).

The controller 140 includes a memory (not shown) that stores algorithms for controlling the operation of helmet components or data corresponding to a program that implements the algorithms and a processor (not shown) that performs the above-described operations using the data stored in the memory.

The storage 140a stores an authentication brain wave, a wear brain wave, a wake word brain wave, and a user registration brain wave. The storage 140a may store signals for the authentication brain waves, wear brain waves, wake word brain waves, and user registration brain waves, and may store a feature point and a feature pattern in the signals together with the signals. The storage 140a may store identification information of each of the personal mobility device and the terminal, and may store the identification information such that the identification information of the personal mobility device corresponds to the authentication brain wave of the personal mobility device. The storage 140a may store various dialogue speeches. For example, various dialogue speeches may include speeches of men and women by generation.

The storage 140a may be a non-volatile memory device such as a cache, a read only memory (ROM), a programmable ROM (PROM), an erasable programmable ROM (EPROM), an electrically erasable programmable ROM (EEPROM), a volatile memory device such as Random Access Memory (RAM), or a storage medium such as a hard disk drive (HDD) and a CD-ROM. However, the present disclosure is not limited thereto. The storage may be a memory implemented in a chip separately provided from the processor described above relation to the controller, and may be implemented in a single chip with the processor.

The personal mobility device 2 may include a communicator, a controller, a power supply, a charge amount detector, a wheel, and the like. When the ignition-on command is received from the helmet, the personal mobility device 2 supplies drive power to various components by turning on the ignition. The personal mobility device 2 blocks the driving power supplied to the various components by turning off the ignition when the ignition-off command is received from the helmet. The personal mobility device 2 may transmit detection information of the riding and departure detector about detecting whether or not the user is getting on and off to the helmet 1. The personal mobility device 2 may transmit information on the battery charge amount of the power supply to the helmet 1.

When the control right is given the user, the personal mobility device 2 controls the driving speed and the driving based on a driving control command inputted by the user, and may automatically control driving on the basis of navigation information when the control right is given the personal mobility device 2. When the control right is not given the user, the personal mobility device 2 does not process the driving control command inputted by the user.

The personal mobility device 2 controls movement based on the target driving speed transmitted from the helmet 1 when the control right is given the helmet 1. At this time, the driving direction of the personal mobility device may be inputted by the user, or may be input from the helmet.

The terminal 3 performs authentication with the helmet. When the authentication with the helmet is successful, the terminal 3 performs a function based on a function control command transmitted from the helmet, and transmits the function execution information to the helmet 1. That is, information on the functions performed in the terminal 3 may be output through the sound output and the image output of the helmet 1.

The terminal 3 may be implemented as a computer or a portable terminal capable of connecting to the helmet 1 through a network. Here, the computer includes, for example, a notebook, a desktop, a laptop, a tablet PC, a slate PC, and the like, on which a WEB Browser is mounted. The portable terminal is a wireless communication device that is guaranteed with portability and mobility: for example, all types of handheld-based wireless communication, such as device a Personal Communication System (PCS), a Global System for Mobile communications (GSM), a Personal Digital Cellular (PDC), a Personal Handyphone System (PHS), a Personal Digital Assistant (PDA), an International Mobile Telecommunication (IMT)-2000, a Code Division Multiple Access (CDMA)-2000, a W-Code Division Multiple Access (W-CDMA), a Wireless Broadband Internet (WiBro), a smart phone, and the like, as well as wireless communication devices; and a wearable device such as a watch, a ring, a bracelet, an ankle bracelet, a necklace, a glasses, a contact lens, or a head-mounted-device(HMD).

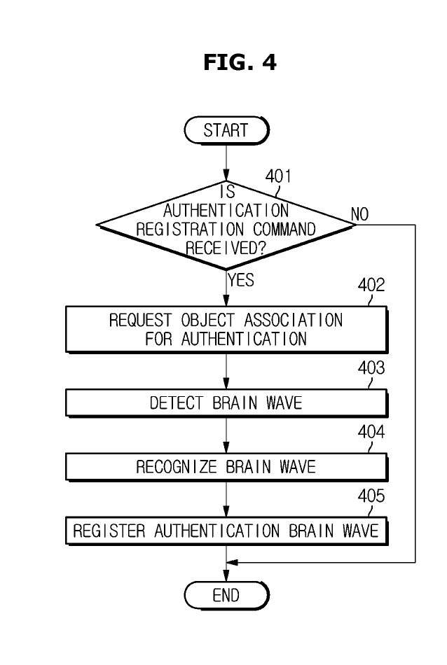

FIG. 4 is a flowchart showing registration of brain waves for authentication of a helmet and a personal mobility device according to embodiments of the present disclosure.

The helmet is configured to, in response to receiving a registration command of an authentication brain wave for authentication with the personal mobility device (401), outputs, through the sound output, a speech of an object association request requesting that an object for authentication with the personal mobility device should be recalled (402), and detects, through the brain wave detector, a brain wave generated from the brain of the user for a predetermined time period from a point of time when the speech corresponding to the object association request is output (403).

The determining that the registration command of the authentication brain wave for authenticating with the personal mobility device is received includes recognizing a speech of the user, determining whether the recognized user's speech is a registration command of an authentication brain wave, and when it is determined that the recognized user's speech is a registration command of an authentication brain wave, determining that the registration command of the authentication brain wave is received.

The determining that the registration command of the authentication brain wave for authenticating with the personal mobility device is received includes determining that a manipulation signal of a registration button for an authentication brain wave of the manipulator provided on the helmet is received. Herein, an object for authenticating with the personal mobility device may include one of a certain object, an image of a certain object, a text of a certain object.

The helmet, in response to receiving the registration command of the authentication brain wave, displays a plurality of images through the image output, requests that one of the plurality of images should be selected, and when one of the plurality of images is selected by the user, displays the selected image to the user, and detects a brain wave through the brain wave detector for a predetermined time period from a point of time at which the selected image is displayed. The helmet recognizes a distinct brain wave from the detected brain waves (404), and registers the recognized brain wave as an authentication brain wave (405). Here, the recognizing of the distinct brain wave of the user includes recognizing a particular point and a particular pattern from signals of the detected brain waves.

For example, the helmet checks a signal of a basic brain wave obtained and stored through an experiment, compares the checked signal of the basic brain wave with a signal of a detected brain wave, identifies a value of the signal of the detected brain wave deviated from a value of the signal of the basic brain wave by a predetermined magnitude, and recognizes the identified value as a particular point, and also the helmet compares a pattern of the basic brain wave signal with a pattern of the detected brain wave signal to identify a pattern of the detected brain wave signal different from that of the basic brain wave signal, and recognizes the identified pattern as a particular pattern. That is, the helmet identifies the amplitude, waveform and frequency of the user's brain waves generated by the wearing of the helmet, and recognizes at least one of a particular pattern and a particular value appearing in at least one of the identified amplitude, waveform and frequency of the brain wave.

The helmet repeats the object association process including an object association request, a brain wave detection, and a brain wave recognition by a predetermined number of times, identifies a feature point and a feature pattern that are common in signals of brain waves recognized by the predetermined number of times of processes, and registers the identified feature point and feature pattern as unique information of the authentication brain wave of the user.

The registering of the authentication brain wave includes storing the authentic brain wave in the storage. The helmet may output a speech that notifies registration of the authentication brain wave.

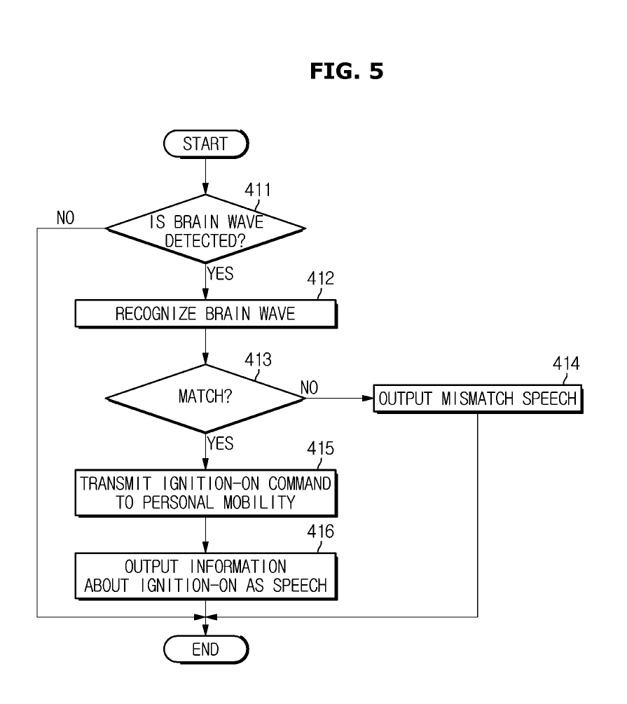

FIG. 5 is a control flowchart for controlling the ignition of a personal mobility device using a helmet according to embodiments of the present disclosure.

As shown in FIG. 5, the helmet, in response to detection of a brain wave through the brain-wave detector 110 (411), recognizes the detected brain wave (412). The recognizing of a brain wave includes identifying the frequency, amplitude, and waveform of the brain wave signal, and recognizing a feature point and a feature pattern in the identified frequency, amplitude, and waveform of the brain wave signal. The helmet may notify the beginning and end of the detection of brain waves through a speech.

The helmet compares the recognized brain wave with the stored brain wave to determine whether the recognized brain wave matches the stored brain waves (413), an when the recognized brain wave does not match the stored brain wave, outputs mismatch information as a speech (414), and when the recognized brain wave matches the stored brain wave, transmits an ignition-on command to the personal mobility device (415) and outputs information about the ignition-on of the personal mobility device as a speech (416).

For example, the helmet compares a recognized brain wave with a stored user registration brain wave, and when the recognized brain wave does not match the stored user registration brain wave, recognizes the user as a unregistered user and outputs a speech indicating an unregistered user, and when the recognized brain wave matches the stored user registration brain wave, recognizes the user as a registered user and transmits an ignition-on command to the personal mobility device, and outputs information about the ignition-on of the personal mobility device as a speech. The comparing between the recognized brain wave and the stored user registration brain wave includes comparing the feature point and the feature pattern of the recognized brain wave signal with the feature point and the feature pattern of the stored user registration brain wave.

In addition, since the brain wave detector may detect the user's brain wave only when the brain wave detector is in contact with the user's head, the helmet determines that the helmet is worn by the user when it is determined that the received brain wave matches the user registration brain wave, and transmits an ignition-on command to the personal mobility device.

As another example, the helmet compares the recognized brain wave with the stored authentication brain wave, and when the recognized brain wave does not match the stored brain wave, recognizes that authentication of the user fails, outputs a speech notifying authentication failure, and when the recognized brain wave matches the stored brain wave, recognized that authentication of the user is successful, transmits an ignition-on command to the personal mobility device, and outputs information about the ignition-on of the personal mobility device as a speech. The comparing between the recognized brain wave and the stored authentication brain wave includes comparing the feature point and feature pattern of the recognized brain wave signal with the feature points and feature pattern of the stored authentication brain wave.

In addition, since the brain wave detector detects the user's brain wave only when the brain wave detector is in contact with the user's head, the helmet determines that the helmet is worn by the user when it is determined that the received brain wave matches the authentication brain wave, and transmits an ignition-on command to the personal mobility device. As another example, the helmet compares a recognized brain wave with a stored wear brain wave, and when the recognized brain wave does not match the stored wear brain wave, outputs a speech notifying that wearing of the helmet fails, and when the recognized brain wave matches the stored wear brain wave, transmits an ignition-on command to the personal mobility device, and outputs information about the ignition-on of the personal mobility device as a speech.

In addition, the helmet may check whether the user is registered and authenticated, and when registration and authentication of the user are successful, compare the recognized brain wave with the stored wear brain wave.

The helmet, in response to no brain wave detected from the brain wave detector, that is, in repose to no brain wave received, may transmit an ignition-off command to the personal mobility device. At this time, the helmet may transmit helmet non-wearing wear information and ignition-off information of the personal mobility device to the terminal such that helmet non-wearing information and ignition- off information of the personal mobility device are output through the terminal.

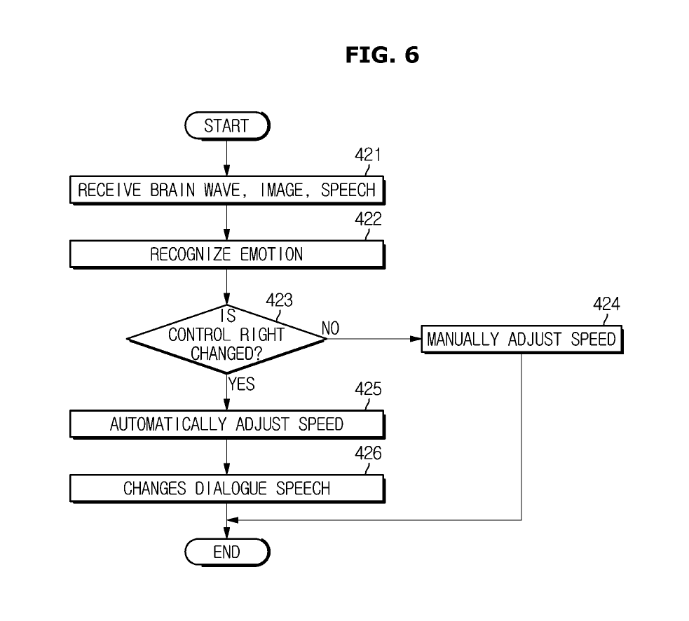

FIG. 6 is a control flowchart for controlling operations of a personal mobility device using a helmet according to embodiments of the present disclosure.

As shown in FIG. 6, the helmet, in response to receiving at least one of a facial image, a speech, and a brain wave of a user (421), recognizes the emotion of the user on the basis of at least one of the facial image, the speech, and the brain wave of the user (422).

In more detail, the helmet recognizes the user's face based on the image inputted through the third image input, recognizes the facial expression of the recognized face, and recognizes the user's emotion based on the recognized facial expression. The helmet recognizes a speech among the sound inputted to the sound input, recognizes the tone, the way of talking, the intonation, and the terminology of the recognized speech, and recognizes the user's emotion based on the recognized tone, way of talking, intonation, and terminology of the speech. The helmet may recognize the user's emotion based on the signal of the recognized brain wave. At this time, signals of brain waves according to emotions may be stored in advance. The helmet recognizes whether the user's emotional state is a pleasure, a joy, a happiness, an anger, an irritation, a tension, a sadness, an excitement, a frustration, an anxiety, or a dissatisfaction, and determines whether to change a control right of the personal mobility device on the basis of the recognized emotional state (423).

The helmet determines that there is no need to change the control right of the person mobility when it is determined that the user's emotional state is one of the joy, the happiness and the pleasure, and maintains the control right of the personal mobility device as being given to the user such that the personal mobility device is driven according to a driving command inputted by the user. That is, the helmet allows the speed of the personal mobility device to be manually adjusted by the user (424). The helmet changes the control right of the personal mobility device from the user to the helmet 1 when it is determined that the user's emotional state is one of the anger, the irritation, the tension, the sadness, the excitement, the frustration, the anxiety, and the dissatisfaction. That is, the personal mobility device having been manually controlled by the user is automatically controlled by the helmet.

That is, in response to determining that the control right of the personal mobility device is changed, the helmet adjusts the driving speed of the personal mobility device with the target driving speed, such that the driving speed is automatically adjusted at the target driving speed or below by recognizing the surrounding environment, such as obstacles and traffic lights, on the basis of image information and brain wave information, and using the recognized information (425). At this time, the helmet transmits information about the driving speed to the personal mobility device such that the speed of the personal mobility device is automatically adjusted.

In addition, when the personal mobility device has an autonomous driving function, the helmet changes the control right of the personal mobility device from the user to the personal mobility device in a case when the emotional state of the user is one of the anger, the irritation, the tension, the sadness, the excitement, and the frustration. At this time, the personal mobility device automatically adjusts the driving speed and the driving direction based on navigation information.

The helmet changes at least one of the tone and the voice of the speech outputted from the helmet and also changes a dialogue response to be output on the basis of the recognized emotion state during execution of the dialogue mode (426). For example, the helmet may change the tone of the speech to a low tone or to a soft voice when the user's emotional state is an anger. The helmet may change the dialogue response to a short response instead of a long response when the user's emotional state is an anger, and may output a response for refreshing the emotional state. For example, the helmet outputs a response speech with content that encourages the user and gives hope when the user's emotional state is a frustration.

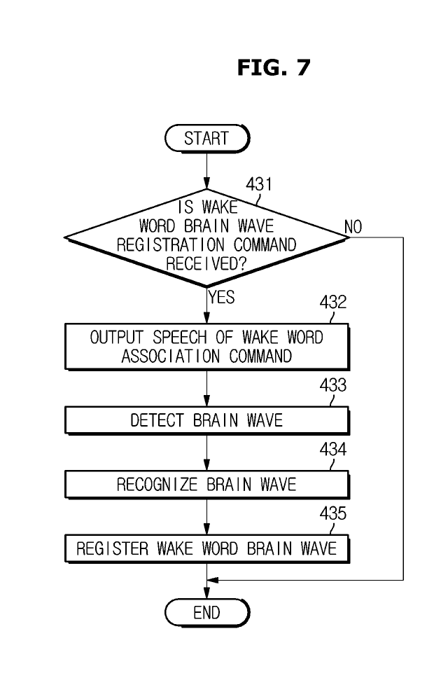

FIG. 7 is a flowchart showing a procedure in which a wake word brain wave are registered in a dialogue mode according to embodiments of the present disclosure.

As shown in FIG. 7, the helmet, in response to receiving a registration command of a wake word brain wave for activating a dialogue mode (431), outputs a speech of an object association request requesting that an object for a wake word should be recalled (432),

The helmet detects, through the brain wave detector 110, a brain wave generated from the brain of the user for a predetermined time period from a point of time when the speech corresponding to the object association request is output (433), recognizes the detected brain wave (434), and registers the recognized brain wave as a wake word brain wave (435).

The determining that the registration command of the wake word brain wave for activating a dialogue mode is received includes recognizing a speech of the user, determining whether the recognized user's speech is a registration command of a wake word brain wave, and when it is determined that the recognized user's speech is a registration command of a wake word brain wave, determining that the registration command of the wake word brain wave is received. The determining that the registration command of the wake word brain wave is received may include determining that a manipulation signal of a registration button for a wake word brain wave of the manipulator provided on the helmet is received. Here, an object for a wake word may include one of a certain object, an image of a certain object, a text of a certain object, and include a name of a dialogue controller (or a dialogue system).

The recognizing of a wake word brain wave includes identifying the amplitude, waveform and frequency of brain wave signals generated before activating a dialogue mode, and recognizing at least one of a particular pattern and a particular value appearing in at least one of the identified amplitude, waveform and frequency of the brain wave signals.

The helmet repeats the object association process including an object association request, a brain wave detection, and a brain wave recognition by a predetermined number of times, identifies a feature point and a feature pattern that are common in signals of brain waves recognized by the predetermined number of times of processes, and registers the identified feature point and feature pattern as information of the wake word brain wave. Here, the registering of the wake word brain wave includes storing the wake word brain wave in the storage.

In addition, the detecting of brain waves includes, in response to receiving a registration command of a wake word brain wave, outputting a wake word utterance request as a speech, and detecting brain waves through the brain wave detector for a predetermined time period from a point of time at which the speech of the wake word utterance request is output. Here, a user's utterance of a wake word is performed during a predetermined time period from the point of time at which the speech of the wake word utterance request is output, and the helmet may detect brain waves generated from a point of time before the utterance of the wake word is performed until the utterance is performed.

Accordingly, the helmet may detect the brain waves generated from the user's brain before the user utters the wake word. In other words, the helmet may detect brain waves generated by the user's thought for performing a dialogue mode.

In addition, the detecting of brain waves includes, in response to receiving the registration command of a wake word brain wave, displaying a plurality of images through the image output, requesting that one of the plurality of images be selected, and when one of the plurality of images is selected by the user, displaying the selected image to the user, and detecting a brain wave through the brain wave detector for a predetermined time period from a point of time at which the selected image is displayed.

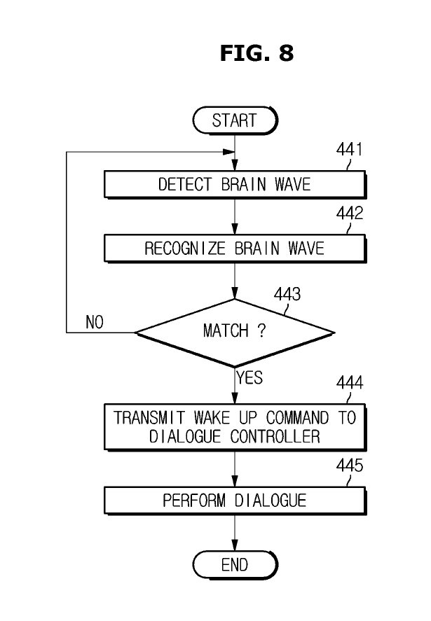

FIG. 8 is a flowchart of entry of a dialogue mode when a dialogue mode is performed using a helmet according to embodiments of the present disclosure.

As shown in FIG. 8, the helmet detects brain waves through the brain wave detector 110 (441), and recognizes the detected brain waves (442). The recognizing of brain waves includes identifying the frequency, amplitude, waveform of brain wave signals, and recognizing a feature point and a feature pattern from the identified frequency, amplitude, and waveform of the brain wave signals.

The helmet compares the recognized brain wave with the stored wake word brain wave, and determines whether the recognized brain wave matches the stored wake word brain wave (443), and when the recognized brain wave does not match the stored wake word brain wave, maintains a sleep mode. The helmet activates a dialogue mode when the recognized brain wave matches the stored wake word brain wave. The comparing of the recognized brain wave with the stored wake word brain wave includes comparing the feature point and feature pattern of the recognized brain wave signal with the feature point and feature pattern of the stored wake word brain wave.

For example, when the stored wake word brain wave is a brain wave corresponding to an object image association and the user recalls an object image corresponding to a wake word, a brain wave associated with the object image is generated from the user. At this time, the helmet recognizes a feature point or feature pattern of the detected brain wave signal, and activates a dialogue mode when the feature point or feature pattern of the recognized brain wave signal matches the feature point or feature pattern of the stored wake word brain wave signal.

In another example, when the stored wake word brain wave is a brain wave corresponding to a text association and the user recalls a text corresponding to a wake word, a brain wave associated with the text is generated from the user. At this time, the helmet recognizes a feature point or feature pattern of the detected brain wave signal, and activates a dialogue mode when the feature point or feature pattern of the recognized brain wave signal matches the feature point or feature pattern of the stored wake word brain wave signal.

As another example, when the stored wake word brain wave is a brain wave corresponding to a name of a dialogue system and the user recalls a name of the dialogue system (that is, a wake word), brain waves are generated from the user from a point of time before the name of the dialogue system is recalled until the recall is performed. At this time, the helmet recognizes a feature point or feature pattern of the detected brain wave signal, and activates a dialogue mode when the feature point or feature pattern of the recognized brain wave signal matches the feature point or feature pattern of the stored wake word brain wave signal. Here, the point of time before the name of the dialogue system is recalled refers to a time when the user thinks before speaking, and the helmet detects brain waves generated before the user makes an utterance.

The activation of the dialogue mode includes waking up the dialogue controller (444). In addition, the activation of the dialogue mode may include activating the sound input and the sound output. The helmet, in response to activation of the dialogue mode, recognizes the user's speech in the sound inputted through the sound input and generates at least one of a response speech and a query speech on the basis of the recognized speech and outputs the generated speech to perform a dialogue with the user (445).

The helmet, in response to a control command of the external device included in the recognized speech, transmits a function control command for controlling at least one of a plurality of functions performed in the external device to the external device on the basis of the control command. For example, the helmet, in response to recognizing a speech ‘Play music’ during execution of a dialogue mode, transmits ‘a music replay command’ to the terminal 3, and outputs a guidance speech for the music replayed to the user. The helmet, in response to recognizing a speech ‘What is the weather today afternoon?’ during execution of a dialogue, may access a weather server to retrieve the weather and output the retrieved result as a speech, or may transmit ‘a weather search command for this afternoon’ to the terminal 3 and output weather information provided from the terminal as a speech. The helmet, in response to recognizing a speech ‘Change the route’ during the dialogue mode, may retrieve a new route from the current location to the destination and output the retrieved route as a speech, and in response to recognizing a speech of a user selecting one of the plurality of routes, output a speech guiding the selected route. The helmet, in response to recognizing a speech of ‘Let me know traffic information’ during execution of a dialogue mode, may access a traffic information providing server to retrieve traffic information from the current location to the destination and output the retrieved result as a speech, and may transmit ‘a traffic information retrieval command’ to the terminal 3, and output traffic information provided from the terminal 3 by a speech.

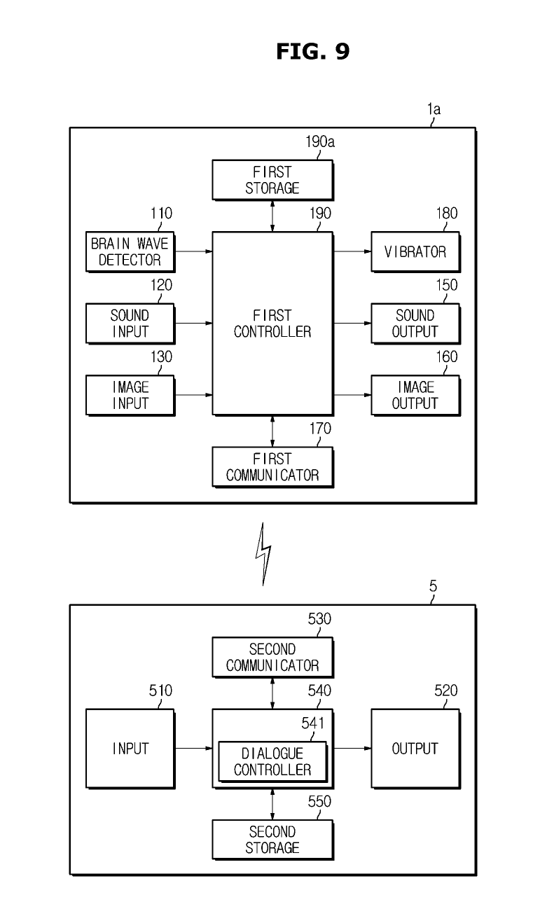

FIG. 9 is another control block diagram of a helmet according to embodiments of the present disclosure.

As shown in FIG. 9, a helmet 1 a includes a brain wave detector 110, a sound input 120, an image input 130, a first controller 190, a first storage 190a, a sound output 150, an image output 160, a communicator 170, and a vibrator 180.

The brain wave detector 110, the sound input 120, the image input 130, the storage 190a, the sound output 150, the image output 160, the communicator 170, and the vibrator 170 according to the another embodiment of the present disclosure are identical to the brain wave detector 110, the sound input 120,the image input 130, the storage 140a, the sound output 150, the image output 160, the communicator 170, and the vibrator 180 according to the example described with reference to FIG. 1, and descriptions thereof will be omitted.

In order to distinguish components having the same name between the helmet 1 a and an external device 5, the term ‘first’ is used to describe parts of the helmet and the term ‘second’ is used to describe parts of the external device 5 .

The controller 190 according to the embodiments of the present disclosure, in response to receiving a registration command of at least one of an authentication brain wave, a wear brain wave, a user registration brain wave, and a wake word brain wave, recognize the detected brain wave in the same manner as the controller 140, and register the authentication brain wave, wear brain wave user registration brain wave, and wake word brain wave. The controller 190 may determine that the user wears a helmet when the recognized brain wave matches at least one of the authentication brain wave, the wear brain wave, and the user registration brain wave, and controls the communicator to transmit an ignition-on command to the personal mobility device, and may control the communicator to transmit an ignition-off command to the personal mobility device when a brain wave is not detected.

The controller 190, in response to determining that the recognized brain wave matches a wake word brain wave, transmits an entry command of a dialogue mode to the external device 5. That is, the controller 190 transmits a wake-up command of the dialogue controller for performing a dialogue. The controller 190 recognizes a speech of the user in sound inputted to the sound input during execution of the dialogue mode and transmits a signal of the recognized speech to the external device 5.

The controller 190, in response to receiving a speech signal corresponding to a response speech or query speech from the external device, controls the sound output to output the received speech signal.

The external device 5 is a device having a function of conducting a dialogue. The external device 5 may be a user terminal having a dialogue function, or may be a dialogue server. For example, a user terminal may have a dialogue function. The external device 5 includes an input 510, an output 520, a second communicator 530, a second controller 540, and a second storage 550.

The input 510 may receive an on-off command of the terminal and a control command for at least one of a plurality of functions, and may receive a command for interoperation and communication with the helmet 1 a. Here, the plurality of functions may be provided as an App (referred to as an “application”).

The output 520 outputs information about the function being performed in the terminal. The output 520 may include a sound output for outputting a speech, sound, and the like, and an image output for displaying an image.

The second communicator 530 performs communication with the helmet 1a.

The second controller 540 controls the operations of the plurality of functions performed in the terminal, recognizes a speech transmitted from the helmet, generates a speech corresponding to at least one of a response speech and a query speech for the recognized speech, and controls output of a signal of the generated speech. When the second controller 540 does not communicate with the helmet 1a, the second controller 540 controls the output 520 to output information about the various functions performed in the terminal through the output 520. The second controller 540, in response to determining that the recognized speech is a function control command, controls such that at least one function is performed based on the determined function control command. The second controller 540 includes a dialogue controller (or a dialogue system) 541 for performing a dialogue with the user, and in response to receiving a wakeup command from the helmet, wakes up the dialogue controller 541 and control entry into a dialogue mode.

The dialogue controller 541 communicates with the helmet 1 a and determines whether a speech signal is received from the helmet when the dialogue mode is activated. When it is determined that a speech signal is received, the dialogue controller 541 performs a speech recognition based on the received speech signal, recognizes the intention of the user from the recognized speech through natural language understanding, determines an action corresponding to the recognized user's intention, generates a response speech and a query speech of a dialogue required to perform the determined action, and transmits a signal for the generated speech. That is, the dialogue controller 541 allows a dialogue speech to be output through the helmet. The dialogue controller 541 may recognize a surrounding circumstance of the user based on brain waves and surrounding images further received from the helmet 1a.

The second storage 550 may store identification information of the helmet 1a and may store information of actions corresponding to the user's intention and the surrounding situation.

When the external device is a dialogue server, the dialogue server, in response to receiving a wake-up command from the helmet, activates a dialogue mode with the helmet and in response to receiving a speech signal from the helmet, performs speech recognition based on the received speech signal, and recognizes the intention of the user from the recognized speech through natural language understanding, determines an action corresponding to the recognized intention of the user, generates a response speech and a query speech required to perform the determined action, and transmits a signal for the generated speech to the helmet. That is, the dialogue server allows a dialogue speech to be output through the helmet.

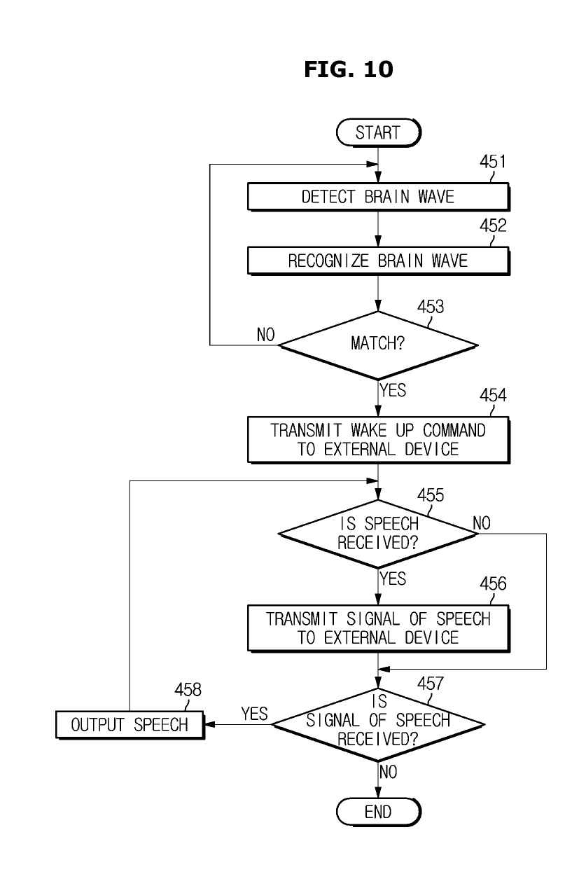

FIG. 10 is another control flowchart of a helmet according to embodiments of the present disclosure.

As shown in FIG. 10, the helmet detects brain waves through the brain wave detector 110 (451), and recognizes the detected brain waves (452). The recognizing of a brain wave includes identifying the frequency, amplitude, and waveform of a signal of the brain wave, and recognizing a feature point and a feature pattern in the identified frequency, amplitude, and waveform of the brain wave signal.

The helmet compares the recognized brain wave with the stored wake word brain wave to determine whether the recognized brain wave matches the stored wake word brain wave (453), and in response to determination that the recognized brain wave matches the stored wake word brain wave, transmits a wake-up command to the external device 5 (454). The comparing between the recognized brain wave and the stored wake word brain wave includes comparing the feature point and the feature pattern of the recognized brain wave signal with the feature point and the feature pattern of the stored wake word brain wave. Here, the transmitting of a wake-up command to the external device includes activating a dialogue mode. The helmet may further include activating the sound input and the sound output.

The helmet, in response to activating a dialogue mode and receiving a speech through the sound input (455), transmits a signal of the received speech to the external device (456).

Then, the helmet, in response to receiving a speech signal from the external device (457), outputs the received speech signal through the sound output as a speech (458). Here, the speech signal may be a signal for at least one of a response speech and a query speech generated and transmitted by the external device.

The disclosed embodiments may be embodied in the form of a recording medium storing instructions executable by a computer. The instructions may be stored in the form of program code and, when executed by a processor, may generate a program module to perform the operations of the disclosed embodiments. The recording medium may be embodied as a computer-readable recording medium. The computer-readable recording medium includes all kinds of recording media in which instructions which can be decoded by a computer are stored, for example, a Read Only Memory (ROM), a Random Access Memory (RAM), a magnetic tape, a magnetic disk, a flash memory, an optical data storage device, and the like.

Although exemplary embodiments of the present disclosure have been described for illustrative purposes, those skilled in the art will appreciate that various modifications, additions and substitutions are possible, without departing from the scope and spirit of the disclosure. Therefore, exemplary embodiments of the present disclosure have not been described for limiting purposes.

As is apparent from the above, the present disclosure can prevent a speech of a user inputted together with a wake word in the beginning of a dialogue from being unrecognized by waking up a dialogue controller before a dialogue with a user is initiated using a brain wave of the user, thereby increasing the dialogue interaction and improving the satisfaction of the user.

In addition, the present disclosure can reduce load and power consumption due to execution of various functions performed in the helmet by obviating the need to execute the dialogue controller in real-time.

In addition, the present disclosure can improve user's convenience by allowing an ignition of the personal mobility device to be turned on by the user's brain wave when the helmet is worn, and allowing an ignition of the personal mobility device to be turned off when the user puts off the helmet or gets off the personal mobility device with the helmet on.

In addition, the present disclosure can start an ignition of the personal mobility device only in a state of a helmet put on.

In addition, the present disclosure can allow a user to easily control at least one of a plurality of functions of a terminal through a dialogue while on the move using the personal mobility device.

In addition, the present disclosure can prevent theft of the personal mobility device by performing authentication between the helmet and the personal mobility device, and prevent others from using the personal mobility when the personal mobility device is stolen.

In addition, the present disclosure can improve the safety of the user and rapidly change the user's feeling by recognizing a user's emotion using at least one of a user's facial expression, speech, and brain waves, and changing an object having a control right of the personal mobility device, changing the tone or voice of a speech outputted by the dialogue controller, and changing content of a dialogue output by the dialogue controller on the basis of the recognized emotion.

As described above, the present disclosure improves the convenience of the interaction between the helmet and the personal mobility device, thereby improving the quality and merchantability of the helmet and personal mobility device, further enhancing the satisfaction of the user.

While the contents of the present disclosure have been described in connection with what is presently considered to be exemplary embodiments, it is to be understood that the disclosure is not limited to the disclosed embodiments, but, on the contrary, is intended to cover various modifications and equivalent arrangements included within the spirit and scope of the appended claims.

What is claimed is:

1. A helmet comprising:
a brain wave detector configured to detect a brain wave of a user;
a sound input configured to receive sound;
a sound output configured to output sound; and
a controller configured to, in response to detecting the brain wave of the user, compare the detected brain wave with a previously stored brain wave, to activate a dialogue mode when the detected brain wave matches the previously stored brain wave, to recognize a speech from the sound received by the sound input, to generate a dialogue speech based on the recognized speech, and to control the sound output so as to output the generated dialogue speech.

2. The helmet of claim 1, further comprising a dialogue controller that is configured to enable a dialogue with the user,
wherein the controller is further configured to transmit a wake-up command to the dialogue controller when activating the dialogue mode.

3. The helmet of claim 2, wherein the previously stored brain wave includes a brain wave generated from a brain of the user before an utterance of a wake word for calling the dialogue controller, the wake word being a target for the dialogue.

4. The helmet of claim 2, wherein the previously stored brain wave includes a brain wave generated from a brain of the user during an object association process for performing the dialogue before an utterance of the user is made.

5. The helmet of claim 1, further comprising a communicator configured to perform communication with a personal mobility device,
wherein the controller is further configured to transmit an ignition-on command to the personal mobility device when the detected brain wave matches the previously stored brain wave.

6. The helmet of claim 5, wherein the controller is further configured to, in response to receiving a registration command for an authentication brain wave of the personal mobility device, control the sound output so as to output a speech corresponding to an object association request, and to register a brain wave recognized from a point of time when the speech corresponding to the object association request is output as the authentication brain wave.

7. The helmet of claim 5, further comprising an image input configured to receive an image of the user,
wherein the controller is further configured to obtain a facial image of the user from the received image, to recognize a facial expression of the user from the obtained facial image, to recognize an emotional state of the user based on the recognized facial expression, and to limit a target speed of the personal mobility device based on the recognized emotional state.

8. The helmet of claim 7, wherein the controller is further configured to recognize the emotional state of the user using the brain wave of the user and a speech of the user.

9. The helmet of claim 1, further comprising a communicator configured to perform communication with a terminal,
wherein the controller, when the recognized speech corresponds to a function control command of the terminal, is further configured to transmit the function control command to the terminal, and in response to receiving function execution information of the terminal, is further configured to control output of the received function execution information.

10. The helmet of claim 9, further comprising a vibrator configured to generate a vibration in response to the transmission of the function control command and the reception of the function execution information.

11. The helmet of claim 1, further comprising:
a main body in which the brain wave detector, the sound input, and the sound output are provided; and
a visor configured to be rotated about a gear axis of the main body.

12. The helmet of claim 11, further comprising an image output provided in the visor, the image output configured to output an image.

13. The helmet of claim 11, further comprising an image output configured to project an image to the visor such that the image is reflected on the visor.

14. The helmet of claim 1, wherein the controller, in response to receiving a registration command for a wake word brain wave for activating the dialogue mode, is further configured to output a speech corresponding to an object association request which corresponds to a wake word, and to register a brain wave recognized from a point of time when the speech corresponding to the object association request is output as the wake word brain wave.

15. A helmet comprising:
a brain wave detector configured to detect a brain wave of a user;
a sound input configured to receive sound;
a sound output configured to output sound;
a communicator configured to communicate with an external device that performs a dialogue; and
a controller configured to, in response to detecting the brain wave of the user, compare the detected brain wave with a previously stored brain wave, to activate a dialogue mode when the detected brain wave matches the previously stored brain wave, to transmit a signal corresponding to the sound received by the sound input to the external device, and to control the sound output so as to output a signal of a speech transmitted from the external device.

16. The helmet of claim 15, wherein the previously stored brain wave includes a brain wave generated from a brain of the user before an utterance of a wake word for calling the dialogue controller, the wake word being a target for the dialogue.

17. The helmet of claim 15, wherein the controller is further configured to, in response to receiving a wake word for activating the dialogue mode, output a speech corresponding to an object association request which corresponds to the wake word, and to register a brain wave recognized from a point of time when the speech corresponding to the object association request is output as a wake word brain wave.

18. The helmet of claim 15, wherein the previously stored brain wave includes a brain wave generated from a brain of the user during an object association process for performing the dialogue before an utterance of the user is made.

19. A method of controlling a helmet, the method comprising:
detecting a brain wave of a user;
comparing the detected brain wave with a previously stored brain wave;
activating a dialogue mode when the detected brain wave matches the previously stored brain wave;
recognizing a speech from sound received by a sound input of the helmet;
generating a dialogue speech based on the recognized speech; and
outputting the generated dialogue speech through a sound output of the helmet.

20. The method of claim 19, wherein the activating of the dialogue mode comprises:
transmitting a wake-up command to a dialogue controller that is configured to enable a dialogue.

21. The method of claim 19, further comprising transmitting an ignition-on command to a personal mobility device when the detected brain wave matches the previously stored brain wave.

22. The method of claim 21, further comprising:
in response to receiving a registration command for an authentication brain wave of the personal mobility device, outputting a speech corresponding to an object association request; and
registering a brain wave recognized from a point of time when the speech corresponding to the object association request is output as the authentication brain wave.

23. The method of claim 19, further comprising:
in response to receiving a registration command for a wake word brain wave for activating the dialogue mode, outputting a speech corresponding to an object association request which corresponds to a wake word; and
registering a brain wave recognized from a point of time when the speech corresponding to the object association request is output as the wake word brain wave.

24. The method of claim 19, further comprising:
when the recognized speech is a function control command of a terminal, transmitting the function control command to the terminal, and
in response to receiving function execution information of the terminal, outputting the received function execution information.
